(19) United States
(12) Patent Application Publication
(10) Pub. No.: US 2024/0182390 A1
(43) Pub. Date: Jun. 6, 2024

(54) METHOD FOR PREPARING PARTIALLY FLUORINATED ALCOHOL

(71) Applicant: Mexichem Fluor S.A. de C.V., San Luis Potosi (MX)

(72) Inventors: Andrew Sharratt, Runcorn (GB); David Grundy, Runcorn (GB); Ira Saxena, Runcorn (GB)

(21) Appl. No.: 18/537,034

(22) Filed: Dec. 12, 2023

Related U.S. Application Data
(62) Division of application No. 17/439,523, filed on Sep. 15, 2021, now Pat. No. 11,897,832.

(30) Foreign Application Priority Data
Mar. 21, 2019 (GB) 1903909.8

(51) Int. Cl. C07C 29/64; C07B 39/00; C07C 29/62

(52) U.S. Cl. CPC C07C 29/64; C07B 39/00; C07C 29/62

(57) ABSTRACT

A method for preparing a partially fluorinated alcohol. comprises reacting an epoxide: wherrein R¹, R², R³ and R⁴ are independently selected from the group comprising H, F, Cl, Br, I, CF₃, alkyl, fluoroalkyl, haloalkyl with a fluorinating agent.

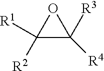

### METHODS

The present invention relates to methods of preparing partially fluorinated alcohols (fluorohydrins) from fluorinated epoxides and preparing fluorinated carbonate esters from fluorohydrins.

Fluorohydrins are useful as solvents and as synthetic building blocks from which various species such as esters, ethers, ketones, aldehydes and acids can be prepared. Of particular interest is their utility in the preparation of fluorinated carbonate esters, which are an important class of materials with significant commercial value. Fluorinated carbonate esters are commonly used without modification as synthetic intermediates and as solvents in electronic devices such as batteries (e.g. lithium ion batteries) and to manufacture products such as lubricants, sealants, and coatings.

The production of fluorohydrins from epoxides is known in the art. For example, Olah described a general method for preparing fluorohydrins by ring opening epoxides with a nucleophilic source of fluoride (G. A. Olah et al, Israel Jr. Chem., 17(1978), 148-149). However, Olah did not extend this work to the preparation of fluorohydrins from fluorinated epoxides.

The ring opening of the fluorinated epoxide, 2,3-epoxy1,1,1-trifluoropropane (TFPO), with various nucleophiles to form fluorohydrins was generally described in a review of the chemistry of TFPO by Uneyama in Jr. Fluorine Chem., 105(2000) 285-293. However, this review was silent on the possibility or likely outcome of attempting to ring open TFPO or indeed any other fluorinated epoxide with nucleophilic fluorinating agents as taught by Olah.

General methods for the production of carbonate esters from alcohols and a carboxylating agent are known in the art, see for example “March's Advanced Organic Chemistry”, M. B. Smith and J. March, 6^{th} edition, page 1276. However, the production of fluorinated carbonate esters from fluorohydrins and carboxylating agents is unknown as are the products of such reactions.

### METHODS OF THE INVENTION

According to a first aspect of the invention there is provided a method for preparing a partially fluorinated alcohol, comprising reacting a fluorinated epoxide:

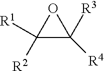

wherein R¹, R², R³ and R⁴ are independently selected from the group comprising H, F, Cl, Br, I, CF₃, alkyl, fluoroalkyl, haloalkyl with a fluorinating agent.

Preferably at least one of R¹ to R⁴ comprises F, CF₃ or fluoroalkyl.

Preferably the fluorinating agent comprises a nucleophilic fluorinating agent. Preferred examples of fluorinating agents include HF and complexes of HF with nitrogen containing species, such as Olah's reagent (HF:Pyridine complex), with urea, or with a tertiary amine.

The method may comprise reacting an epoxide of 3,3,3-Trifluoropropene (1243zf) with HF and/or Olah's reagent to form CF₃CH(OH)CH₂F.

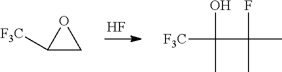

The method may comprise reacting an epoxide of 1,3,3,3-Tetrafluoropropene (1234ze) with HF and/or Olah's reagent to form CF₃CH(OH)CHF₂.

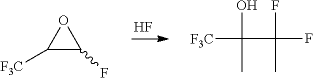

The method may comprise reacting an epoxide of 1,1,1,4,4,4-hexafluoro-2-butene (1336mzz) with HF and/or Olah's reagent to form CF₃CH(OH)CHF(CF₃).

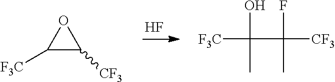

The method may comprise reacting an epoxide of 1,1,3,3,3-Pentafluoropropene (1225zc) with HF and/or Olah's reagent to form CF₃CH(OH)CF₃.

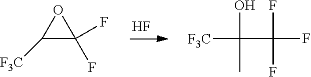

### COMPOUNDS & COMPOSITIONS OF THE INVENTION

According to a second aspect of the invention there is provided compounds with the structure

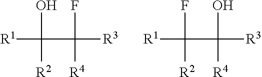

wherein R¹, R², R³ and R⁴ are independently selected from the group comprising H, F, Cl, Br, I, CF₃, alkyl, fluoroalkyl, haloalkyl, with the provision that the compound is not 1,1,1,3-tetrafluoropropan-2-ol.

The compounds of the second aspect of the invention may be used in the preparation of a carbonate ester.

According to a third aspect of the invention there is provided a method for preparing a partially fluorinated carbonate ester with the structure

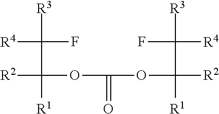

comprising reacting a fluorohydrin

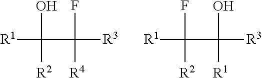

wherein R¹, R², R³ and R⁴ are independently selected from the group comprising H, F, Cl, Br, I, CF₃, alkyl, fluoroalkyl, haloalkyl with COX₂, wherein X is selected from the group comprising —F, —Cl, —OCH₃, —OCCl₃, imidazole, succinimidyl.

Preferably 2 equivalents (on a molar basis) of the fluorohydrin are used per 1 equivalent of COX₂.

Alternatively, 1 equivalent of the fluorohydrins of this invention maybe used with 1 equivalent of an alcohol species (a branched or linear monohydric/polyhydric alcohol) to prepare asymmetric carbonate esters.

The compounds produced in a method according to the third aspect of the invention are covered by the fourth aspect of the invention. According to the fourth aspect of the invention there is provided a compound with the structure

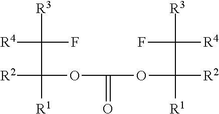

wherein R¹, R², R³ and R⁴ are independently selected from the group comprising H, F, Cl, Br, I, CF₃, alkyl, fluoroalkyl, haloalkyl.

The compounds of the fourth aspect of the invention may be also used as a battery solvent component (e.g. in a lithium ion battery). Here the compounds are found to be beneficial as a result of their physical properties, electrochemical stability, compatibility with battery components such as battery electrodes (cathodes and anodes) including electrodes comprising carbon and silicon, lithium containing electrolyte salts, separators, binders, current collectors and low flammability.

The compounds of the fourth aspect of the invention may also be used with other solvents and additives such as other linear and cyclic carbonate esters.

Preferably when used as a solvent the composition comprises an electrolyte salt. Preferred examples of electrolyte salts include lithium-based electrolytes such as those selected from the group comprising lithium hexafluorophosphate (LiPF₆), lithium triflate (LiSO₃CF₃), lithium bis(fluorosulfonyl)imide (Li(FSO₂)₂N) and lithium bis(trifluoromethanesulfonyl)imide (Li(CF₃SO₂)₂N).

The compounds of the second aspect of the invention may be used in the preparation of a (more highly) fluorinated derivative. One or more of the R groups may be substituted by fluorine. In the process the R groups to be altered by fluorination are preferably selected from the group comprising H, Cl, Br, I.

Depending on the nature of the R group(s) being modified the preparation process for the fluorinated derivative may comprise a multi-stage process; preferably a two-stage process. In a preferred two stage process a first stage is the modification of the targeted R group(s) to a (different) halogen group, preferably to a chlorine group (with a suitable chlorinating agent such as chlorine); in a second stage the chlorine group is modified to a fluorine group (with a suitable fluorinating agent such as HF or a metal fluorine salt, such as NaF, KF). It will be appreciated that where the R group(s) targeted already comprises a halogen, other than fluorine, a two-stage process with substitution of the halogen with chlorine may not be necessary.

Thus compounds

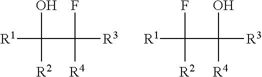

wherein at least 2 of R¹ to R⁴ independently comprises H, Cl, Br, I may be converted to (more highly) fluorinated derivatives

In the fluorinated derivatives preferably at least 2 and more preferably at least 3 of R¹ to R⁴ independently comprises F, CF₃ or a fluoroalkyl. Preferably at least 1 of R¹ to R⁴, and more preferably 1 of R¹ to R⁴ independently comprises H. Most preferably 1 of R¹ to R⁴ comprises CF₃, two of R¹ to R⁴ comprise F and one of R¹ to R⁴ comprises H. Most preferably the fluorinated derivative comprises hexfluroroisopropanol.

A preferred reaction pathway occurs for the compound

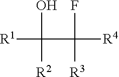

wherein in the preferred alternatives
R¹ is —CF₃, R² is H and both R³ and R⁴ are H.
R¹ is —CF₃, R² is H; one of R³ and R⁴ is H, one of R³ and R⁴ is F.

This preferred pathway is shown below.

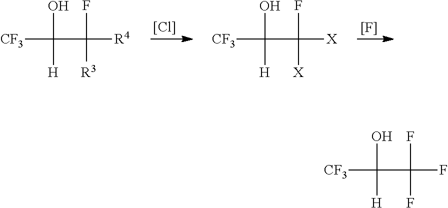

X is either F or Cl.

The epoxides useful in the first aspect of the invention may prepared from a fluorinated alkene. According to a fifth aspect of the invention there is provided a method for preparing a partially fluorinated epoxide, comprising reacting a fluorinated alkene:

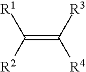

wherein R¹, R², R³ and R⁴ are independently selected from the group comprising H, F, Cl, Br, I, CF₃, alkyl, fluoroalkyl, haloalkyl with an oxidising agent.

Preferably at least one of R¹ to R⁴ comprises F, CF₃ or fluoroalkyl.

Preferred examples of oxidising agent include air, oxygen and oxygen containing compounds such as peroxides, per-salts and compounds of oxygen with other elements such as hypohalites. Preferably the oxidising agent comprises a hypohalite such as chlorite.

Preferably, the compound reacted with the oxidising agent is a tetrafluoropropene. Most preferably, one of R¹ and R² is —CF₃ and one of R³ and R⁴ is —F. Thus, the tetrafluoropropene is 1,3,3,3-Tetrafluoropropene (1234 ze) or 2,3,3,3-Tetrafluoropropene (1234yf).

According to a sixth aspect of the invention there is provided a method for preparing a fluorohydrin comprising the fifth and the first aspects of the invention.

According to a seventh aspect of the invention there is provided a method for preparing a partially fluorinated ether with the structure

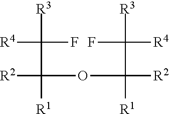

comprising reacting a fluorohydrin with the structure

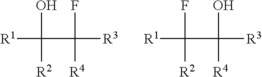

wherein R¹, R², R³ and R⁴ are independently selected from the group comprising H, F, Cl, Br, I, CF₃, alkyl, fluoroalkyl, haloalkyl.

According to an eighth aspect of the invention there is provided a compound with the structure

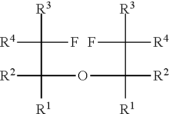

wherein R¹, R², R³ and R⁴ are independently selected from the group comprising H, F, Cl, Br, I, CF₃, alkyl, fluoroalkyl, haloalkyl.

According to a ninth aspect of the invention there is provided a composition comprising a compound of the eighth aspect of the invention.

The compound of the eighth aspect of the invention or the composition according to the ninth aspect of the invention may be used as a solvent, for example, in battery applications.

The compound of the eight aspect of the invention or the composition according to the ninth aspect of the invention may be used as a coolant, for example, as an immersive coolant.

Also provided is a method for preparing a partially fluorinated ether with the structure

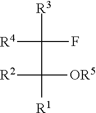

comprising reacting a fluorohydrin with the structure

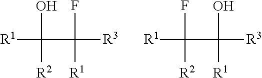

wherein R¹, R², R³, R⁴ are independently selected from the group comprising H, F, Cl, Br, I, CF₃, alkyl, fluoroalkyl, haloalkyl and R⁵ is independently selected from the group CF₃, alkyl, fluoroalkyl, perfluoroalkyl, haloalkyl perfluorohaloalkyl.

Preferably the ether synthesis occurs via acid catalysed dehydration of the fluorohydrin.

Alternatively the ether synthesis occurs via one or more of the following techniques:
i. Alkoxy-de-halogenation—reaction of an alkyl halide with a fluorohydrin, preferably under basic conditions;
ii. Alkoxy-de-sulphonyloxy-substitution—reaction of an fluorohydrin sulphate with an alkoxide or fluorohydrin alkoxide;
iii. Hydro, alkoxy-de-diazo-disubstitution—reaction of a fluorohydrin with a diazo compound;
iv. Alkoxy-de-hydroxylation—dehydration of two alcohols to yield an ether with e.g. concentrated sulphuric acid;
v. Hydroxy or alkoxy-de-alkoxylation—transetherification of a (fluorohydrin)ether with an alcohol or fluorohydrin; and/or
vi. Alkoxy-de-hydroxylation—reaction of an alcohol or fluorohydrin with an oxonium compound.

Additionally provided is a compound with the structure

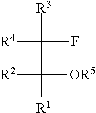

Further provided is a composition comprising a compound with the structure

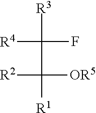

The compound or the composition may be used as a solvent, for example, in battery applications.

The compound or the composition may be used as a coolant, for example, as an immersive coolant.

The invention will now be illustrated with reference to the following non-limiting examples.

### EXAMPLES

### Example 1—Ring Opening of an Epoxide with Olah's Reagent

The following steps were followed.
The reactor was charged with Olah's reagent (70% HF:Pyridine, 5 ml) and cooled in an ice batch with stirring.
2,3-epoxyl,1,1-trifluoropropane (TFPO) (3.4 g) was then added dropwise.
At the end of the addition the reaction mixture was allowed to warm up to room temperature; stirring was continued for 48 hours.
After 48 hours the reaction mixture was quenched with ice.
Salt was added, and the product extracted with diethyl ether (3×5 ml). The diethyl ether extracts were combined, washed with saturated potassium bicarbonate solution and water before being dried over anhydrous sodium sulphate. Diethyl ether was removed in vacuo to yield the desired product as a clear, colourless liquid boiling point 91-93° C. The identity of this product was confirmed by NMR spectroscopy.

### Example 2—Ring Opening of 2,3-epoxy-1,1,1,3-tetrafluoropropane with Olah's Reagent

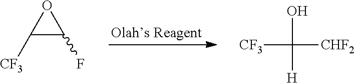 [SMILES: FC1OC1C(F)(F)F ; OC(C(F)F)C(F)(F)F]

2,3-epoxy-1,1,1,3-tetrafluoropropane was ring opened using the following procedure:
A 100 ml Hastalloy C pressure reactor was charged with Olah's reagent (70% HF:Pyridine, 25 g).
After sealing, the contents of the reactor were cooled to 20° C. with stirring.
2,3-epoxy-1,1,1,3-tetrafluoropropane (11 g) was then added.
After this addition was complete the reaction mixture was heated to 50° C. and stirred for 168 hours.
After 168 hours the reaction mixture was quenched with ice and saturated sodium chloride solution (22 ml) added.
The product was extracted from this mixture with diethyl ether.
The diethyl ether extracts were combined, washed with saturated potassium bicarbonate solution and then water before being dried over anhydrous sodium sulphate. The identity of the product was confirmed by NMR spectroscopy.

### Example 2a—Ring Opening of 2,3-epoxy-1,1,1,3-tetrafluoropropane with Olah's Reagent

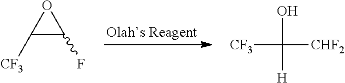 [SMILES: FC1OC1C(F)(F)F ; OC(C(F)F)C(F)(F)F]

2,3-epoxy-1,1,1,3-tetrafluoropropane was ring opened using the following procedure:
A 100 ml Hastalloy C pressure reactor was charged with Olah's reagent (70% HF:Pyridine, 25 g).
After sealing, the contents of the reactor were cooled to 20° C. with stirring.
2,3-epoxy-1,1,1,3-tetrafluoropropane (10.6 g) was then added.
After this addition was complete the reaction mixture was heated to 80° C. and stirred for 43 hours.
After 43 hours a sample of the reaction mixture was analysed by GCMS and it was found that all the feed epoxide had reacted.
After cooling the reaction mixture was quenched with ice and saturated sodium chloride solution (22 ml) added.
The product was extracted from this mixture with diethyl ether.
The diethyl ether extracts were combined, washed with saturated potassium bicarbonate solution and then water before being dried over anhydrous sodium sulphate. The identity of the product was confirmed by NMR spectroscopy.

### Example 3—Ring Opening of 2,3-epoxy-1,1,1-trifluoro-2-(trifluoromethyl)Propane with Olah's Reagent

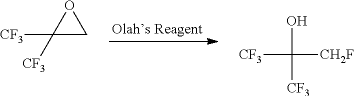

2,3-epoxy-1,1,1-trifluoro-2-(trifluoromethyl)propane was ring opened using the following procedure:
A 100 ml Hastalloy C pressure reactor was charged with Olah's reagent (70% HF:Pyridine, 16.5 g).
After sealing, the contents of the reactor were cooled to 20° C. with stirring.
2,3-epoxy-1,1,1-trifluoro-2-(trifluoromethyl)propane (10 g) was then added.
After this addition was complete the reaction mixture was heated to 50° C. and stirred for 160 hours.
After 160 hours the reaction mixture was quenched with ice and saturated sodium chloride solution (22 ml) added.
The product was extracted from this mixture with diethyl ether.
The diethyl ether extracts were combined, washed with saturated potassium bicarbonate solution and then water before being dried over anhydrous sodium sulphate. The identity of the product was confirmed by NMR spectroscopy.

### Example 4—Preparation of di-(1,1,1,3-tetrafluoropropyl) Carbonate with Phosgene

Di-(1,1,1,3-tetrafluoropropyl) carbonate was synthesised using the following procedure:
A three necked round bottom flask was cooled to 0° C. under an inert atmosphere.
Phosgene solution (15% by weight in toluene, 50 mL of solution) was added and stirred.
A mixture of 1,1,1,3-tetrafluoropropan-2-ol (18.42 g) and pyridine (11.02 g) was added to the solution dropwise, and the temperature of the solution was monitored to ensure it did not rise above 10° C.
The solution was allowed to warm to room temperature and stirred for 48 hours.
The product was filtered to remove pyridinium salts and the solvent was removed in vacuo to yield the crude product.
The crude product was distilled under atmospheric conditions to yield di-(1,1,1,3-tetrafluoropropyl) carbonate as a yellow oil (7.08 g, 35% yield).

### FIGURES

The Figures illustrates the results of various spectroscopic analytical techniques carried out on some of the reaction products from the Examples.

FIG. 1 shows a ¹⁹F NMR spectrum of the reaction product of 2,3-epoxyl, 1,1-trifluoropropane (TFPO) with Olah's reagent.

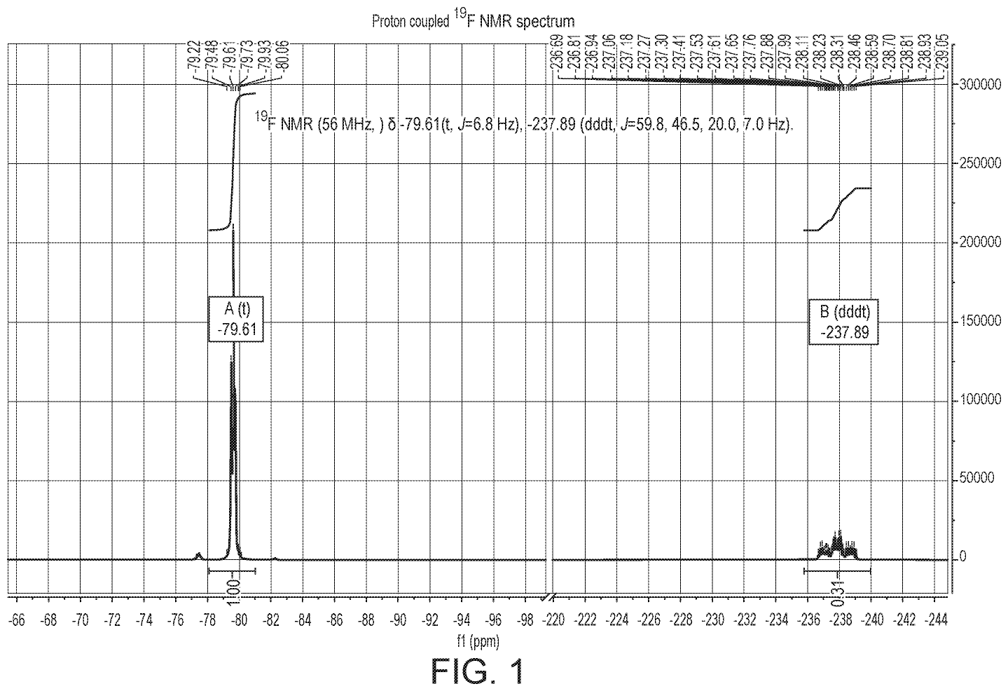

FIG. 2a shows a ¹⁹F NMR spectrum of the reaction product of 2,3-epoxy-1,1,1,3-tetrafluoropropane ring opening with Olah's reagent.

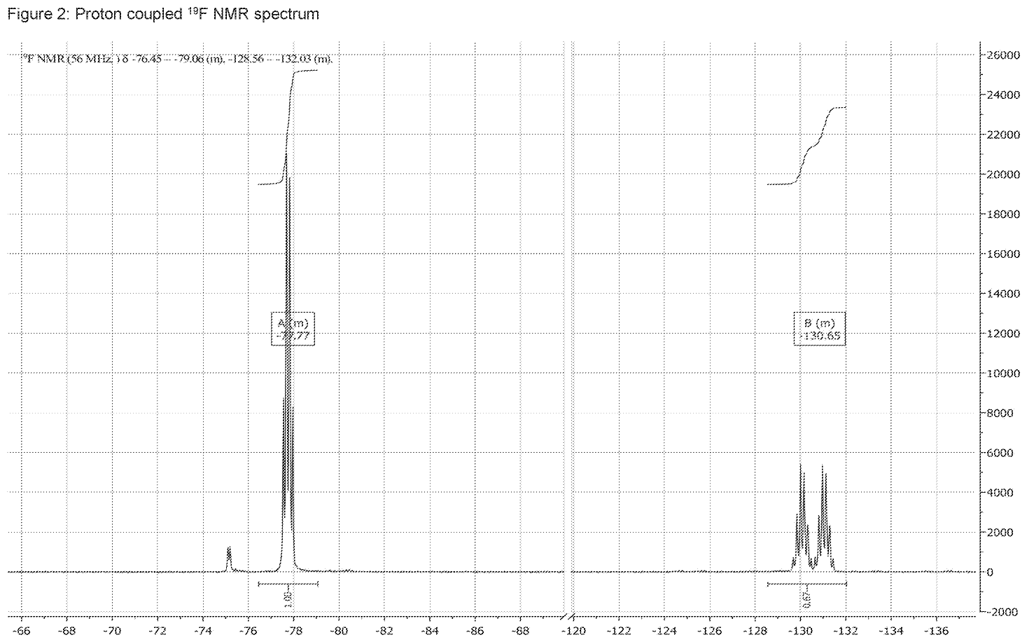

FIG. 2b shows a proton coupled ¹⁹F NMR spectrum of the reaction product of 2,3-epoxy-1,1,1,3-tetrafluoropropane ring opening with Olah's reagent.

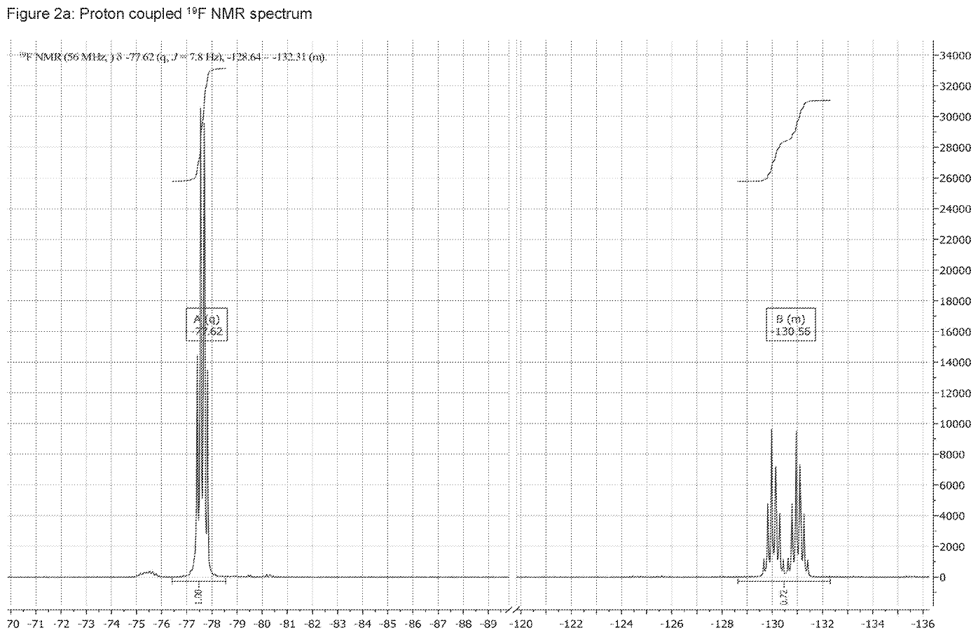

FIG. 2c shows a proton decoupled ¹⁹F NMR spectrum of the reaction product of 2,3-epoxy-1,1,1,3- tetrafluoropropane ring opening with Olah's reagent.

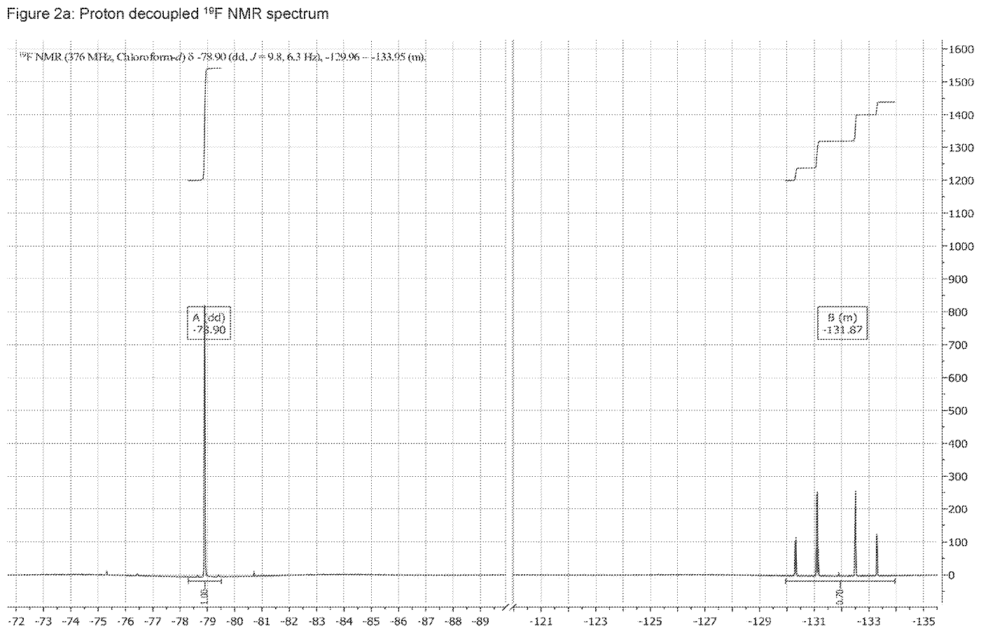

FIG. 3 shows a ¹⁹F NMR spectrum of the reaction product of 2,3-epoxy-1,1,1-trifluoro-2-(trifluoromethyl)propane) ring opening with Olah's reagent.

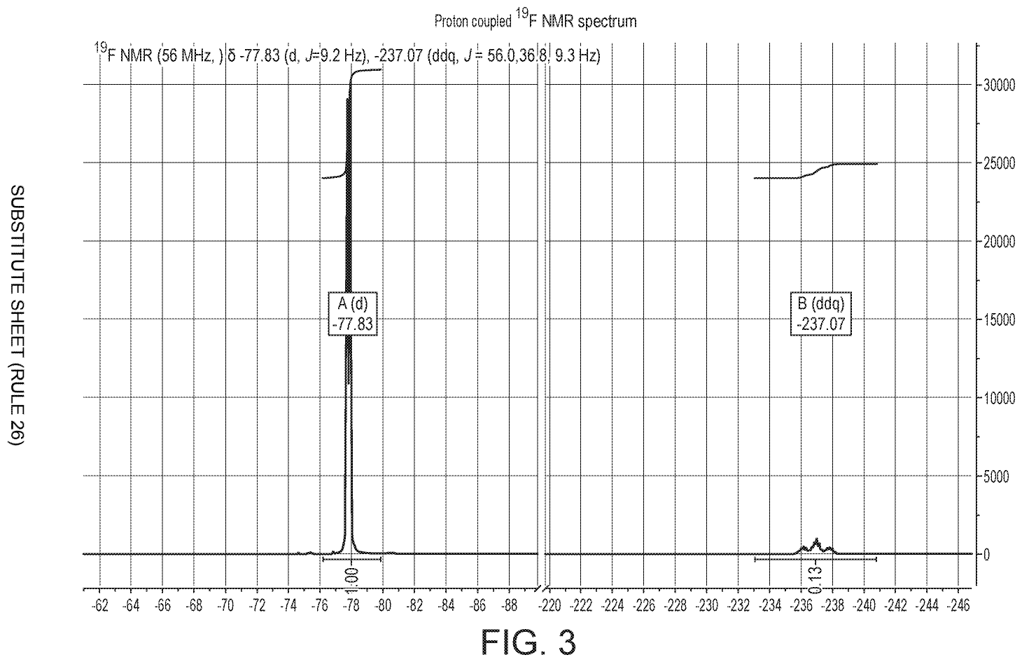

FIG. 4 shows a ¹⁹F NMR spectrum of the reaction product of 1,1,1,3-tetrafluoropropan-2-ol with phosgene, consistent with that of the product di-(1,1,1,3-tetrafluoropropyl) carbonate.

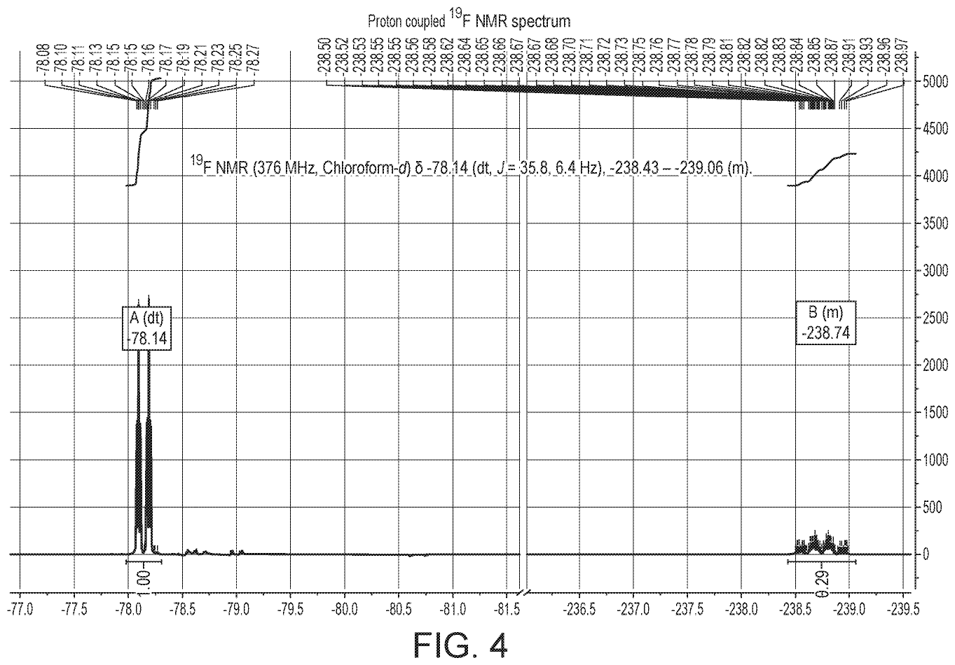

1.-8. (canceled)

9. Compounds with either of the following structures 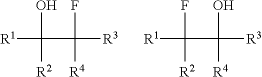
wherein R¹, R², R³ and R⁴ are independently selected from the group consisting of H, F, Cl, Br, I, CF₃, alkyl, fluoroalkyl, and haloalkyl, with the provision that the compound is not 1,1,1,3-tetrafluoropropan-2-ol.

10. A compound of claim 9, formed by the process comprising reacting an epoxide: 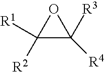
wherein R¹, R², R³ and R⁴ are independently selected from the group consisting of H, F, Cl, Br, I, CF₃, alkyl, fluoroalkyl, and haloalkyl;
with a fluorinating agent.

11. A method for preparing a partially fluorinated carbonate ester with the structure 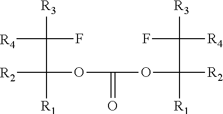
comprising reacting a fluorohydrin having either of the following structures 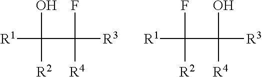
wherein R¹, R², R³ and R⁴ are independently selected from the group consisting of H, F, Cl, Br, I, CF³, alkyl, fluoroalkyl, and haloalkyl
with COX₂, wherein X is selected from the group consisting of fluorohydrin —F, —Cl, —OCH₃, —OCCl₃, imidazole, and succinimidyl.

12. A compound with the structure 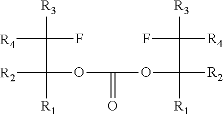
wherein R¹, R², R³ and R⁴ are independently selected from the group consisting of H, F, Cl, Br, I, CF₃, alkyl, fluoroalkyl, and haloalkyl.

13. A composition comprising the compound with the structure according to claim 12

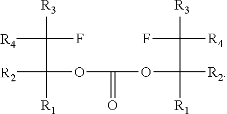

14. A solvent composition comprising the compound according to claim 12.

15. A solvent composition comprising the composition according to claim 13.

16. A solvent composition according to claim 14, wherein the composition comprises an electrolyte comprising a lithium salt, selected from the group comprising lithium hexafluorophosphate (LiPF₆), lithium triflate (LiSO₃CF₃), lithium bis(fluorosulfonyl)imide (Li(FSO₂)₂N) and lithium bis(trifluoromethanesulfonyl)imide (Li(CF₃SO₂)₂N).

17. A method for preparing a fluorinated compound, comprising reacting a compound having either of the following structures: 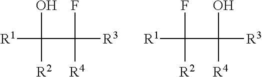
wherein at least 2 of R¹ to R⁴ independently comprise H, Cl, Br, or l;
with a fluorinating agent.

18. The method according to claim 17, wherein in the fluorinated compound at least 2 of R¹ to R⁴ independently comprise F, CF₃ or a fluoroalkyl.

19. The method according to claim 17, wherein in the fluorinated compound at least 1 of R¹ to R⁴ comprises H.

20. The method according to claim 17, wherein the fluorinated compound comprises hexafluoroisopropanol.

21. A method for preparing a partially fluorinated epoxide, comprising reacting a partially fluorinated alkene: 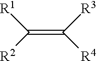
wherein R¹, R², R³ and R⁴ are independently selected from the group consisting of H, F, Cl, Br, I, CF₃, alkyl, fluoroalkyl, and haloalkyl;
with an oxidising agent.

22. The method according to claim 21, wherein the oxidising agent is selected from the group consisting of air, oxygen and oxygen containing compounds.

23. The method according to claim 21, wherein the oxidising agent comprises a hypohalite.

23. (canceled)

24. The method for preparing a partially fluorinated alcohol, comprising a method for preparing a partially fluorinated epoxide according to claim 21 and reacting the partially fluorinated epoxide with a fluorinating agent.

25. The method for preparing a partially fluorinated alcohol, comprising a method for preparing a partially fluorinated epoxide according to claim 21, reacting the partially fluorinated epoxide with a first fluorinating agent to form a compound having at least either of the following structures: 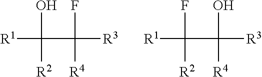
wherein at least 2 of R¹ to R⁴ independently are H, Cl, Br, or l; and
reacting the compound having the at least either of the above structures with a second fluorinating agent.

26. A method for preparing a partially fluorinated ether with the structure 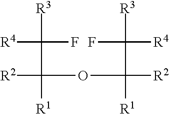
comprising reacting a fluorohydrin having either of the following structures 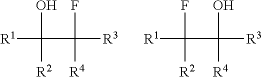
wherein R¹, R², R³ and R⁴ are independently selected from the group consisting of H, F, Cl, Br, I, CF₃, alkyl, fluoroalkyl, or haloalkyl.

27. A compound with the structure 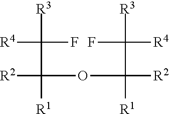
wherein R¹, R², R³ and R⁴ are independently selected from the group consisting of H, F, Cl, Br, I, CF₃, alkyl, fluoroalkyl, and haloalkyl.

28. A composition comprising the compound with the structure according to claim 27

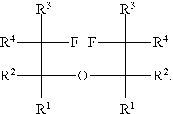

29. A solvent composition comprising the compound according to claim 27.

30. A coolant composition comprising the composition according to claim 28.

31. A method for preparing a partially fluorinated ether with the structure 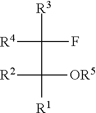
comprising reacting a fluorohydrin having either of the following structures 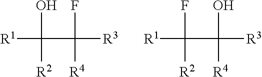
wherein R¹, R², R³, R⁴ are independently selected from the group comprising H, F, Cl, Br, I, CF₃, alkyl, fluoroalkyl, haloalkyl and R⁵ is independently selected from the group consisting of CF₃, alkyl, fluoroalkyl, perfluoroalkyl, haloalkyl and perfluorohaloalkyl.

32. A compound with the structure 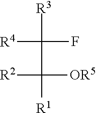
wherein R¹, R², R³, R⁴ are independently selected from the group comprising H, F, Cl, Br, I, CF₃, alkyl, fluoroalkyl, haloalkyl and R⁵ is independently selected from the group consisting of CF₃, alkyl, fluoroalkyl, perfluoroalkyl, haloalkyl and perfluorohaloalkyl.

33. A composition comprising the compound with the structure according to claim 32

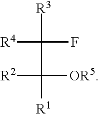

34. A solvent composition comprising the compound according to claim 32.

35. A coolant composition comprising the composition according to claim 33.

36. A method according to claim 21, wherein the alkene comprises: a tetrafluoropropene from the group consisting of 1,3,3,3-Tetrafluoropropene (1234ze) and 2,3,3,3-Tetrafluoropropene (1234yf); or a pentafluoropropene consisting of 1,1,3,3,3-Pentafluoropropene (1225zc).
